(19) United States
(12) Patent Application Publication
(10) Pub. No.: US 2003/0073109 A1
(43) Pub. Date: Apr. 17, 2003

(54) Methods for preparing improved enzyme variants

(76) Inventors: Jae-Gu Pan, Daejeon (KR); Heung-Chae Jung, Daejeon (KR); Yong-Sung Kim, Daejeon (KR)

Correspondence Address: FITZPATRICK CELLA HARPER & SCINTO, 30 ROCKEFELLER PLAZA, NEW YORK, NY, 10112, US

(21) Appl. No.: 10/185,990

(22) Filed: Jun. 28, 2002

Related U.S. Application Data
(63) Continuation-in-part of application No. 09/395,881, filed on Sep. 14, 1999, now abandoned.

(30) Foreign Application Priority Data
Mar. 15, 1999 (KR) 99-8677

(51) Int. Cl. C12Q 1/68; C12N 15/74; C12N 1/21

(52) U.S. Cl. 435/6; 435/252.3; 435/471

(57) ABSTRACT

The present invention relates to a high throughput screening method for preparing a variant of catalytic polypeptide capable of catalyzing a chemical reaction. The method of selecting a bacterium comprising a nucleic acid sequence encoding a polypeptide capable of catalyzing a chemical reaction from a plurality of candidate bacteria comprises the following steps of: (a) generating of a pool of nucleic acids by introducing at least one nucleotide change into the target nucleic acids encoding the polypeptide capable of catalyzing the desired chemical reaction, (b) constructing library vectors to be transformed into a host cell after subcloning said pool of candidate nucleic acids into a surface display vector wherein said resulting vectors direct expression of fusion polypeptides of display motifs and candidate polypeptides and said fusion polypeptides are to be anchored to the surface of said bacteria, (c) transforming said library vectors into bacteria, (d) expressing each of said fusion polypeptides on the surface of host bacteria, and (e) selecting a bacterium expressing a desired polypeptide on the basis of said host bacterial phenotypic changes, or expressing a desired polypeptide on the basis of visual changes of said products.

This application is a continuation-in-part of U.S. patent application Ser. No. 09/395,881, filed on Sep. 14, 1999.

### BACKGROUND OF THE INVENTION

1. Field of the Invention

The present invention relates generally to the field of high-throughput protein evolution. Particularly, it concerns a high-throughput screening method for preparing a polypeptide showing improved catalytic performance and the population of transformants prepared therefrom.

2. Description of the Prior Art

In general, directed evolution of a catalytic polypeptide (i.e., enzyme or catalytic antibody) refers to the acquisition of its variant with desired properties by serial processes of random mutagenesis and screening. Directed evolution is to acquire new traits of a catalytic polypeptide needed for industrial applications unlike the properties of polypeptides easily obtained from the extracts of various organisms in nature, such as the increase in catalytic activity itself; change in substrate specificity; change in selectivity for photo-active substrates; the increase of stability against temperature, organic solvents or highly concentrated salts (Kuchner and Arnold, Trends Biotechnol., 1997, 15, 523-530,). Hence, directed evolution of a catalytic polypeptide can be delineated as a continuous process of the following 3 steps: 1) to determine the properties of a catalytic polypeptide needed to improve, 2) to construct a catalytic polypeptide library by appropriate mutagenesis and to express them as a soluble form in the appropriate host cells, 3) to acquire the final polypeptide by utilizing various selection and screening methods.

For the above-mentioned evolution of a catalytic polypeptide, the construction of libraries of mutated polypeptide variants is a prerequisite. The necessity of genetic diversity for the improvement of a protein, i.e., the library of polypeptide variants can be constructed by using such methods as error-prone PCR, point mutation using mutator host cells, combinatorial cassette mutagenesis or DNA shuffling (Stemmer, Nature, 1994, 370, 389-391).

However, the limiting factor in directed protein evolution lies not in the construction of the genetic libraries itself but in how to screen and select the appropriate enzyme variants with the desired properties from the given libraries within a short period of time.

The up-to-date methods for screening and selecting a catalytic polypeptide variants are based on the use of phenotypic selection or screening in either solid phase or microtitier well plates as follows: 1) an evolution method of antibacterial degrading enzyme having improved antibiotic-resistant which is a method of selecting colonies formed in agar plates containing increased concentration of antibiotics (Stemmer, Proc. Natl. Acad. Sci., 1994, 91, 10747-10751); 2) a chromogenic substrate method for visual screening (Zhang et al., Proc. Natl. Acad. Sci., 1997, 94, 4504-4509; Moore and Arnold, Nature Biotechnol., 1996, 14, 458-467); and 3) a positive selection method using auxotrophic host cells (Yano et al., Proc. Natl. Acad. Sci., 1998, 95, 5511-5515). However, unfortunately, the utility of phenotypic selections are limited to the isolation of a catalytic polypeptide for reactions that are of direct biological relevance or can be indirectly linked to a selectable phenotype. Alternatively, each host cell expressing polypeptide mutants may be screened directly by measuring catalytic activity of them. Screening can be performed on colonies growing on a agar substrate surface, which relies on substrate of desired catalytic activity

With the importance of the above-mentioned screening and selection steps being more emphasized recently, the more effective methods have been developed as described hereunder: 1) a selection method of faster growing, subtilisin-secreting Bacillus subtilis clones cultured in hollow fiber membranes using bovine serum albumin (BSA) as the only nitrogen source (Naki et al., Appl. Microbiol. Biotechnol., 1998, 43, 230-234), 2) a novel screening method using the infectivity of phage (Spada et al., J. Biol. Chem., 1997, 378, 445-456), 3) a screening method using the difference between genomic DNA and expressed enzymes in water-oil suspension (Tawfik and Griffiths, Nature Biotechnol., 1998, 16, 652-656).

In general, small polypeptide libraries composed of 10³-b 10⁶ distinct variants can be screened by first expressing them in the cytoplasm of host cells and growing each clone separately and then using conventional assay for detecting clones that exhibit desired catalytic activity of variants. To detect activity, the cells are lysed to release the expressed polypeptides and lysates are transferred to the microtiter well plates, which are then measured using chromogenic or fluorescently labeled substrates. However, it is difficult to screen large libraries consisting of tens of millions or billions of clones. Further, in order to avoid expression biases resulting from the folding and solubility efficiency of each of polypeptide variants, protein concentration should be measured and then the specific activity of clones should be corrected, which is an obstacle for high throughput screening of intracellularly expressed enzyme variants. This expression normalization process requires another tier of screening processes.

Furthermore, for evolution of toxic enzymes such as protease, lipase, phospholipase, esterase, etc. it is difficult to intracellularly express them, thus secretion strategy is of choice. However, secretion method for high-throughput screeing (HTS) of enzymes can be complicated by a number of factors. First, it is also necessary to normalize the expression of target enzyme variants. Often is the case of screening enzyme variants showing increased folding and solubility efficiency of enzyme variants as of the case of intracellularly expressed libraries. Very small fraction of screened variants may be true positive clones showing high specific activity. Second, secreted variants are limited to ones compatible to secretion machinery of host cells. This can lower total number of library size. Third, specific protein transporters are necessary for secretion of target enzyme variants. So intracellular enzymes can not be transported through host cell membrane without help of above mentioned specific transporter systems. Lastly, total number of visual screening on solid surface is limited because the secreted enzyme variants can diffuse distantly and thus be cross-contaminated between adjacent colonies before discriminating them on the solid surface.

Georgiou et al. recently invented the periplasmic expression of binding proteins including antibodies or their fragments or enzymes for screening them with high affinity or catalytic activities (Georgiou et al, WO 02/34886). They provided a method of screening a bacterium expressing desired antibodies of fragments or enzyme variants in the periplasmic space, wherein the target ligand or substrates are diffused into the periplasmic space through the disturbed outer membrane. However, it has still problems of expression normalization and secretion compatibility of expressed antibodies or enzyme variants. Host cells are also restricted to Gram negative bacteria which have the periplasmic bag for polypeptide library. To detect target clone, labeled ligand for antibodies or substrate for enzymes are limited to molecules comprising molecular weight of greater than 600 Da and less than about 30,000 Da.

The screening of very large protein library has been accomplished by a variety of techniques that rely on the display of proteins on the surface of viruses or cells (Ladner et al. 1993). The fundamental characteristics of surface display technologies is that proteins engineered to be anchored on the external surface of biological particles (viruses or cells) are directly accessible for interacting to target molecules without the need for lysing the cells.

Even though phage display allow to select a good binding partner from large library (10¹⁰-10¹¹) to the target ligand, it is not practical to screen catalytic polypeptide (enzyme or catalytic antibody) library displayed on the surface of phage particles. There is no apparent way to physically link in a quantitative manner a phage particle displaying a certain enzyme clone with the outcome of multiple catalytic turnovers resulting in the accumulation of reaction product. This signal amplification is essential for the high throughput screening of enzyme library.

### SUMMARY OF THE INVENTION

The inventors of this invention have been exploring faster and more convenient screening methods of catalytic polypeptide variants and able to find that the screening and selection procedure can be carried out faster and more conveniently when using a surface display system that enables foreign proteins to express stably and effectively on the given microbial surface.

In one aspect, the invention provides a method of selecting a bacterium comprising a nucleic acid sequence encoding a polypeptide capable of catalyzing a chemical reaction from a plurality of candidate bacteria comprising the following steps of: (a) generating of a pool of nucleic acids by introducing at least one nucleotide change into the target nucleic acids encoding the polypeptide capable of catalyzing the desired chemical reaction, (b) constructing library vectors to be transformed into a host cell after subcloning said pool of candidate nucleic acids into a surface display vector wherein said resulting vectors direct expression of fusion polypeptides of display motifs and candidate polypeptides and said fusion polypeptides are to be anchored to the surface of said bacteria, (c) transforming said library vectors into bacteria, (d) expressing each of said fusion polypeptides on the surface of host bacteria, and (e) selecting a bacterium expressing a desired polypeptide on the basis of said host bacterial phenotypic changes.

In another aspect, the invention provides a method of selecting a bacterium comprising a nucleic acid sequence encoding a polypeptide capable of catalyzing a chemical reaction from a plurality of candidate bacteria comprising the following steps of: (a) generating of a pool of nucleic acids by introducing at least one nucleotide change into the target nucleic acids encoding the polypeptide capable of catalyzing the desired chemical reaction, (b) constructing library vectors to be transformed into a host cell after subcloning said pool of candidate nucleic acids into a surface display vector wherein said resulting vectors direct expression of fusion polypeptides of display motifs and candidate polypeptides and said fusion polypeptides are to be anchored to the surface of said bacteria, (c) transforming said library vectors into bacteria, (d) expressing each of said fusion polypeptides on the surface of host bacteria, and (e) selecting a bacterium expressing a desired polypeptide on the basis of visual changes of said products.

In another aspect of the invention, the catalytic polypeptide anchored to the external surface of the bacterium is further defined as enzymes including oxidoreductase, transferase, hydrolase, lyase, isomerase, and ligase. In one embodiment of the invention, the catalytic polypeptide is catalytic antibody which is an immunoglobulin polypeptide capable of catalyzing a chemical reaction.

In yet another aspect, the invention is further defined as surface display of target catalytic polypeptides is accomplished by fusing them to an adequate surface display motif from a variety of microorganisms including bacteriophage, bacteria, yeast, or spores. In one embodiment of the invention, surface display motif could be an outer membrane protein from Gram-negative bacteria including Lpp (outer membrane lipoprotein), PAL (peptidoglycan-associated lipoprotein), OmpA, OmpC, OmpF, Inp(ice-nucleation protein), Pilin (pili protein), flagellin (flagellar protein), etc. Among them, the ice-nucleation protein is chosen because it could not provoke problems with destabilization of outer membrane after insertion of target fusion polypeptides into the outer membrane and display of them to the surface of the outer membrane. Potentially, any Gram negative bacterium could be used with the invention, including, for example, an E. coli bacterium. Still further, cell wall proteins from Gram positive bacteria could be used as a surface display motif. Potentially, any Gram positive bacterium could be used with the invention, including, for example, an B. subtilis bacterium. Still further, catalytic polypeptides could be displayed on yeast cell surface by using cell wall proteins such as Aga1p, Cwp1p, Cwp2p, Flo1p, Sed1p, Tip1p, Tir1p, and etc. Still further, microbial spores such as bacterial spores, yeast spores, or fungal spores could be used for display of catalytic polypeptides. Bacteriophages, still further, could be used for display of catalytic polypeptides. Still further particularly, surface display of ‘library’ of catalytic polypeptide is defined as display of library on the surface of particular microorganism including bacteriophage, bacteria, yeast, or spores.

In still yet another aspect of the invention, a candidate bacterium is further defined as a bacterium displaying polypeptide variants showing desired catalytic properties. Still further, selection of a candidate bacterium is based on the phenotypic change of host bacterial cells. In one embodiment of this invention, this measurable physical link between phenotype of the host cells and surface-displayed polypeptide variants is performed by ‘cell surface display technology’. The phenotypic change may be still further defined as based on the different growth rate. The different growth rate is based on the different colony size on the semisolid surface. The semisolid surface may be an agar plate. In certain embodiments of the invention, different growth rate is defined as the change of optical density in liquid culture. Still further, growth is defined as supported by products released from catalysis of substrate.

In still yet another aspect of the invention, selection of a candidate bacterium is based on the visual change of products. Still further, visual change of products is defined as clearance around the bacterial colonies. Clearance may be from the hydrolysis of the substrate. In a preferred embodiment of the invention, the hydrolysis of the substrate is further defined as hydrolysis of polymer substrate, such as starch, cellulose, xyllan, pullulan, levan, dextran, agar, gelatin, casein, and etc. In a preferred embodiment of the invention, visual change of products is further defined as change of color around the bacterial colonies. Visual change of products may further fluorescence around the bacterial colonies. Still further, selection or screening could be carried out with high throughput screening instruments such as fluorescent activated cell sorting (FACS) flow cytometer according to change of fluorescent properties of the substrate during reaction.

In still yet another aspect, the invention comprises providing a population of bacteria. In one embodiment of the invention, the population of bacteria is further defined as collectively capable of expressing a plurality of candidate catalytic polypeptides. In yet another embodiment of the invention, the population of bacteria is obtained by a method comprising the following steps of: (a) generating of a pool of nucleic acids by introducing at least one nucleotide change into the target nucleic acids encoding the polypeptide capable of catalyzing the desired chemical reaction, (b) constructing library vectors to be transformed into a host cell after subcloning said pool of candidate nucleic acids into a surface display vector wherein said resulting vectors direct expression of fusion polypeptides of display motifs and candidate polypeptides and said fusion polypeptides are to be anchored to the surface of said bacteria, (c) transforming said library vectors into bacteria, and (d) expressing each of said fusion polypeptides on the surface of host bacteria.

Finally, the object of this invention is to provide a high throughput screening method of improved catalytic polypeptide variants, and another goal of this invention is to provide transformants so prepared as in the above methods.

### BRIEF DESCRIPTION OF THE DRAWINGS

FIG. 1 is an ion chromatogram of hydrolyzed products of CMC by CMCase.

FIG. 2 is a cleavage map of a recombinant plasmid pYSK3 used in this invention.

FIG. 3 is a schematic diagram of plasmid construction and DNA shuffling procedure of the CMCase gene. The cel and mcel mean the CMCase and mature CMCase gene, and X and H show XmaI and HindIII, respectively.

FIG. 4 is a picture of colonies of transformed E. coli JM 109/pYSK3 displaying CMCase on their surface (magnification, ×2.5). Parent CMCase (A) and the shuffled CMCase library (B) from the first round of mutagenesis are shown after colonies were formed after 72 hr of growth on M9 minimal agar plate at 37° C. containing 0.5%(wt/vol) CMC as the sole carbon source. (C) transformants of the shuffled CMCase library were spread and grown for 24 hr on M9 gloucose agar at 37° C.

FIG. 5 is a picture of Congo Red staining of E. coli JM109 colonies displaying evolved CMCase variants on LB ampicillin agar plates with o0.5%(wt/vol) CMC. (A) Colonies selected from M9 CMC plates showing outgrowth. (B) Colonies reandomly chosen from a library of transformants grown on LB ampicillin plates. Control colonies are shown in each photograph. The first control colony is JM109(pUSK3), the second is JM109(pEIN229) and the third is JM109(pKK223-3). All other colonies were selected as CMCase variants.

FIG. 6 is a graph that shows the enzyme activity of whole cells of variants produced according to the method proposed in this invention.

FIG. 7 is a graph that shows specific enzyme activity of enzyme variants with increased activities produced according to the method of this invention, expressed in separated forms within cells.

FIG. 8 is a photograph of Western blot of the soluble fractions of corresponding CMCase variants in same order in FIG. 6 and FIG. 7.

FIG. 9 is a schematic diagram of contruction of pJHC12 vector for display of lipase via INP display motif.

FIG. 10 is (A) a photograph of library colonies of TG1 cells picked on tributyrin LB plates and (B) their corresponding whole cell lipase activities in 96 well plates.

FIG. 11 is a graph that shows whole cell lipase profile of 120 selected colonis from 25,000 library colonies in 96 well plates.

FIG. 12 is a graph that shows whole cell lipase activities of finally selected 4 mutant colonies.

### DETAILED DESCRIPTION OF PREFERRED EMBODIMENTS

The present invention circumvents the limitations of the prior art and provides a novel tool for the screening of very large polypeptide libraries. In particular, the invention overcomes deficiencies in the prior art by providing a very high throughput approach for isolating a polypeptide that catalyzes a desired substrate for production of desired product via ‘display library screening’.

The present invention is characterized by the HTS method for preparing improved catalytic polypeptide variants and also characterized by a microbial transformants produced according to methods described above. The specific details of this invention are as follows

In one aspect, the invention provides a method of selecting a bacterium comprising a nucleic acid sequence encoding a polypeptide capable of catalyzing a chemical reaction from a plurality of candidate bacteria comprising the following steps of: (a) generating of a pool of nucleic acids by introducing at least one nucleotide change into the target nucleic acids encoding the polypeptide capable of catalyzing the desired chemical reaction, (b) constructing library vectors to be transformed into a host cell after subcloning said pool of candidate nucleic acids into a surface display vector wherein said resulting vectors direct expression of fusion polypeptides of display motifs and candidate polypeptides and said fusion polypeptides are to be anchored to the surface of said bacteria, (c) transforming said library vectors into bacteria, (d) expressing each of said fusion polypeptides on the surface of host bacteria, and (e) selecting a bacterium expressing a desired polypeptide on the basis of said host bacterial phenotypic changes.

In another aspect, the invention provides a method of selecting a bacterium comprising a nucleic acid sequence encoding a polypeptide capable of catalyzing a chemical reaction from a plurality of candidate bacteria comprising the following steps of: (a) generating of a pool of nucleic acids by introducing at least one nucleotide change into the target nucleic acids encoding the polypeptide capable of catalyzing the desired chemical reaction, (b) constructing library vectors to be transformed into a host cell after subcloning said pool of candidate nucleic acids into a surface display vector wherein said resulting vectors direct expression of fusion polypeptides of display motifs and candidate polypeptides and said fusion polypeptides are to be anchored to the surface of said bacteria, (c) transforming said library vectors into bacteria, (d) expressing each of said fusion polypeptides on the surface of host bacteria, and (e) selecting a bacterium expressing a desired polypeptide on the basis of visual changes of said products.

The technology of surface display in which organism displays on its surface the desired proteinaceous substance such as peptide and polypeptide has wider application fields depending on the types of protein displayed or host organism (Georgiou et al., 1993, 1997; Fischetti et al., 1993; and Schreuder et al., 1996). Such conventional surface display technology has been developed by use of several unicellular organisms such as bacteriophage, bacteria, yeast, mammalian cells, or spores.

The gene of a polypeptide to be displayed is contained in host organism and thus the host can be selectively screened using the characteristics of the protein displayed, thereby obtaining the desired gene from the selected host with easiness. Therefore, such surface display technology can guarantee a powerful tool on molecular evolution of protein (see WO 9849286; and U.S. Pat. No. 5,837,500).

For instance, phage displaying on its surface antibody having desired binding affinity is bound to immobilized antigen and then eluted, followed by propagating the eluted phage, thereby yielding the gene coding for target antibody from phage (U.S. Pat. No. 5,837,500). The biopanning method described above can provide a tool to select target antibody by surface displaying antibody library on phage surface in large amount and comprises the consecutive steps as follows: (1) constructing library; (2) surface displaying the library; (3) binding to immobilized antigen; (4) eluting the bound phage; finally (5) propagating selected clones.

The technology of phage surface display has been found to be useful in obtaining the desired monoclonal variant form enormous library (e.g., 10⁶-10⁹ variants) and thus applied to the field of high-throughput screening of antibody. Antibody has been used in various fields such as therapy, diagnosis, analysis, etc. and thus its demand has been largely increased. In this context, there has been a need for novel antibody to have binding affinity to new substance or catalyze biochemical reaction. The hybridoma technology to produce monoclonal antibody has been conventionally used so as to satisfy the need. However, the conventional method needs high expenditure and long time for performance whereas the yield of antibody is very low. In addition to this, to screen novel antibody, more than 10¹⁰ antibody libraries is generally used, as a result, the hybridoma technology has been thought to be inadequate in finding antibody exhibiting new binding property.

Many researches has focused on novel methods which is easier and more effective that the biopanning method described above and then developed novel technologies performed in such a manner that libraries are displayed on surface of bacteria or yeast and then cells displaying target protein is sorted with flow cytometry in a high-throughput manner. According to the technology, antigen labeled with fluorescent dye is bound to surface-displaying cell and the antibody having the desired binding affinity is isolated with flow cytometry capable of analyzing more than 10⁸ cells a hour. Francisco, et al., have demonstrated the usefulness of microbial display technology by revealing that surface-displayed monoclonal antibody could be concentrated with flow cytometry at rate of more than 10^{5,} finally more than 79% have been proved to be the desired cells (Daugherty et al., 1998).

However, high throughput screening method for catalytic polypeptide is more difficult to screen out from large phage library than that of binding polypeptides. In this invention, by using cell surface display technology, very high throughput screening for catalytic polypeptide is provided. Surface display of the library of catalytic polypeptide could be accomplished on the surface of a variety of organisms including Gram-negative or Gram positive bacterial cells, yeast cells, mammalian cells, microbial spores, or bacteriophages. The invention is not limited by the host organism.

In the method for improving a polypeptide of interest, the step of constructing a gene library by mutation of wild type gene of protein of interest by means of: DNA shuffling method (Stemmer, Nature, 370: 389-391(1994)), StEP method (Zhao, H., et al., Nat. Biotechnol., 16: 258-261 (1998)), RPR method (Shao, Z., et al., Nucleic acids Res., 26: 681-683 (1998)), molecular breeding method (Ness, J. E., et al., Nat. Biotechnol., 17: 893-896 (1999)), ITCHY method (Lutz S. and Benkovic S., Current Opinion in Biotechnology, 11: 319-324 (2000)), error prone PCR (Cadwell, R. C. and Joyce, G. F., PCR Methods Appl., 2: 28-33 (1992)) and point mutagenesis (Sambrook et al., Molecular Cloning: A Laboratory Manual, Cold Spring Harbor, N.Y., 1989).

From this invention, it was found that a catalytic polypeptide is an enzyme or a catalytic antibody. In a preferred embodiment of this invention, the catalytic polypeptide is an enzyme. In one example of invention, a hydrolase is an effective enzyme, such as carboxylmethylcellulase (CMCase) or lipase. However, the enzyme is not limited to CMCase or lipase.

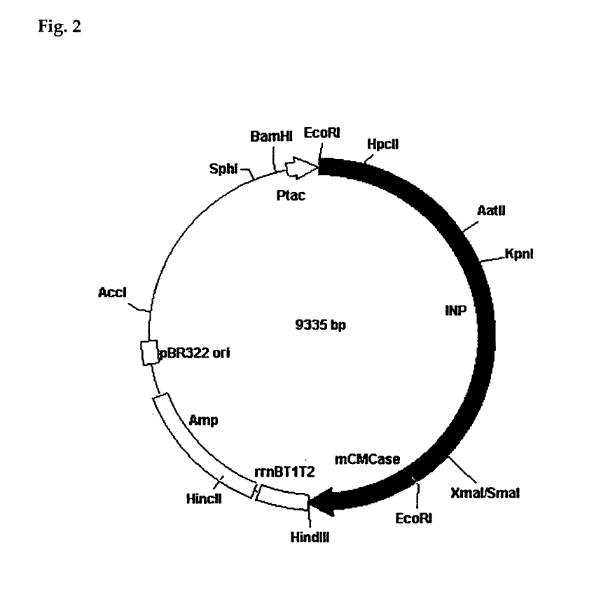

The displaying vector used in this invention contains a replication origin, a gene resistant to antibiotics, useful restriction enzyme sites, a gene that encodes a protein displayed on cell surface, an inserting site for an enzyme-encoding-gene located proximal to a gene that codes for a cell-surface displaying protein motif. In this invention, it is strongly desired to use pYSK3 (KCTC 0584BP) vector and its cleavage map is shown in FIG. 2.

The pYSK3 is a displaying vector that can utilize an Ice Nucleation active Protein (INP) surface display system invented and patent-registered by the inventors of this invention (Korean patent No. 185335), and the display system is a very stable and effective technique without such defects of either directly affecting the structure of outer cell membrane or decreasing the viability of host cells as shown in previous systems (Georgiou et al., Protein Engineering, 1996, 9, 239-247; Jung, et al., Nature Biotechnology, 1998, 16, 579-580). By utilizing pYSK3 vector, the enzyme library can be displayed on bacterial surface and the enzyme established on bacterial surface can get an easier access to its substrate and colonies can be visually identified because each colony can form a halo on an agar plate. When the substrate for CMCase is a polymer of polysaccharides, the above-mentioned advantages can be more obvious. When an enzyme's substrate, which can grow on products produced by the reaction of relevant enzymes, is used as the only nutrient, the substrate cannot be utilized by host cells but the microbes can grow depending on either the amount of a substrate-hydrolyzing enzyme displayed on the cell surface or the activity of an enzyme, and the growth rate of microbes can be accelerated by the increased activities of enzyme variants so that they can form larger colonies on minimal agar plates containing polymer substrates, and the process of screening and selection can be also more rapidly performed with visual distinction. The microbial host recommended to use in this invention is Gram negative bacteria.

In high throughput screening and selection method of the invention, selecting a bacterium expressing a desired polypeptide is based on the phenotypic changes of said host organisms. The most commonly occurred phenotypic change is growth of the host organism. Growth is supported only by products coming from result of catalytic reaction of the substrate. Growth can be measured on solid agar surface containing said substrates by colony size, or in liquid culture media by increase of optical density of the culture. The said substrate may be sole carbon source, or sole nitrogen source, or sole phosphate source, or sole sulphate source, or sole metal source. In preferred embodiment, the said substrate may be sole carbon and energy source. In an example of the invention, carboxymethylcellulose (CMC) used as a substrate is degraded by carboxymethylcellulase (CMCase) and further, degree of hydrolysis of CMC is dependent on the specific activity of CMCase. Still further, when the library of CMCase is displayed on E. coli cells, the growth can be distinguished by colony size on semisolid agar plate containing carboxylmethylcellulose (CMC) as its sole carbon source or the halo size formed by CMCase on minimal agar plates. The increased catalytic activities can be monitored directly on culture media by color reaction without lysis of host microbes, which allowed the inventors to screen the large library of CMCase with very high throughput.

In still further, the screening of surface displayed library can be performed by visual changes of said substrate. In one embodiment of the invention, when the lipase library was displayed on the surface of E. coli and the population of transformants was spread on the minimal agar plate containing tributyrin as a sole carbon and energy source, large colony size and halo formation was observed around the colonies showing higher specific lipase activity. Thus the change of visual properties of the substrate is a good screening tool for surface-displayed library of catalytic polyptides.

### EXAMPLES

The following examples illustrate various aspects of the present invention herein but are not to be construed to limit claims in any manner whatsoever. In particular, CMCase and lipase are only examples of catalytic polypeptides and the high-throughput screening method for their variants described in this invention can be applied to other catalytic polypeptides, preferably to hydrolytic enzymes.

### Materials and Methods

Bacterial Strains, Plasmids, and Culture Conditions

E. coli JM109 (recA1supE44 endA1 hsdR17 gyrA96 relA1 thi Δ(lac-proAB)F9 [traD36 proAB 1 lacI qlacZ DM15]) and TG1 (supE hsdΔ5 thi Δ(lac-proAB)F' [traD36 proAB lacI^{q} lacZΔM15]) were used as a host cell for DNA manipulations and gene expression. pKK223-3 containing a tac promoter (Amersham Pharmacia Biotech, Uppsala, Sweden) was used so that the expression of the Inp fusion protein or foreign proteins could be induced with isopropyl-b-D-thiogalactoside (IPTG) for high-level gene expression in E. coli. pSSTS110 (Jung et al., 1998, Nat. Biotechnol. 16:576-580) was employed for surface display of CMCase and lipase. A CMCase gene (endo-b-1,4-glucanase, EC 3.2.1.4) from B. subtilis BSE616 (GenBank accession number D01057) was originated from plasmid pUBS101 (Park, et al., 1991. Agric. Biol. Chem. 55:441-448). A lipase gene from Pseudomonas fluorescens SIK W1 was originated from pHOPE (Ahn et al., 1999, J. Bacteriol., 181:1847-1852). Recombinant E. coli cells were grown at 37° C. in Luria-Bertani (LB) medium containing yeast extract, 5 g/liter; tryptone, 10 g/liter; and NaCl, 5 g/liter. When appropriated, ampicillin was added to a final concentration of 100 μg/ml. Cell growth was determined by measuring optical density of the culture at 600 nm (OD600 ) with an Ultraspec 2000 spectrometer (Amersham Pharmacia Biotech).

HPLC Analysis of CMC Hydrolysates

The products of CMC hydrolysis by CMCase were analyzed using high-pH anion-exchange chromatography (Dionex, Sunnyvale, Calif.). Separation cellopentaose (G5), cellotetraose (G4), cellotriose (G3), and cellobiose (G2), was accomplished by using a CarboPac PA1 analytical column (Dionex, 4 by 250 mm) and a CarboPac PA1 guard column (4 by 50 mm) with a mobile phase containing a mixture of eluent 1 (deionized water), eluent 2 (200 mM NaOH), and eluent 3 (200 mM NaOH, 1 M sodium acetate) at a flow rate of 1.0 ml/min. A PAD system with a gold electrode was used for detection of carbohydrates. A Dionex Advanced Computer Interface (ACI) model III was used for data ac-quisition with Dionex AI-450 software, version 3.32. For hydrolysis of cellopentaose (Sigma, St. Louis, Mo.), a reaction mixture containing 100 μl of 10 mg of cellopentaose per ml, 20 μl of purified CMCase (0.09 mg/ml), and 80 μl of 50 mM sodium phosphate buffer (pH 5.5) was incubated for 120 min at 37° C. To analyze the CMC hydrolysate, a reaction mixture containing 100 μl of 10 mg of CMC per ml, 20 μl of 3×10⁸ cells displaying an evolved CMCase variant (2R52) and 80 μl of 50 mM sodium phosphate buffer (pH 5.5) were incubated for 180 min at 37° C. The cells were removed by centrifugation before high-pressure liquid chromatography (HPLC) analysis.

Random Mutagenesis and Catalytic Polypeptide Library Display

Random mutagenesis of the CMCase or lipase gene was performed by DNA shuffling as described previously (Stemmer, W. P. 1994. Nature 370:389-391; Zhao, H., and F. H. Arnold. 1997, Nucleic Acids Res. 25:1307-1308). Briefly, for example of CMCase library generation and its display, the substrates for the shuffling reaction were 1.3-kb double-stranded-DNA PCR products derived from pYSK3 by using a recombinant Taq DNA polymerase (TaKaRa Shuzo Co., Shiga, Japan) with two primers, SEQ ID No. 1 and 2, which are annealed to the outside of CMCase gene. PCR conditions were 30 cycles of 94° C. for 30 s, 50° C. for 30 s, and 72° C. for 60 s. After digestion of about 5 μg of the DNA substrates with DNase I (Boehringer Mannheim, Dusseldorf, Germany), 50- to 200-bp fragments were recovered on a 2% agarose gel and reassembled by PCR without primers by using a PCR program of 60 cycles of 94° C. for 30 s, 50° C. for 30 s, and 72° C. for 65 s. A 50-fold dilution of PCR assembled products was used for the final production of single PCR products of the correct size (1.3 kb) with 30 pmol of each primer and 30 additional PCR cycles (94° C. for 60 s, 55° C. for 60 s, and 72° C. for 60 s). For this PCR amplification, two internal primers, SEQ ID No. 3 and 4, which anneal just inside of the first primer set, were used. After successful reassembly and amplification, reactions were verified by 0.8% agarose gel electrophoresis, and the shuffled products were purified with a Wizard PCR Prep Kit (Promega, Madison, Wis.), digested with terminal restriction enzymes, XmaI and HindIII, and subcloned into pYSK3. This process produced the plasmids containing the mutated CMCase genes that were fused to the end of the Inp gene. Library plasmids were used to transform competent E. coli JM109 or TG1 cells by a high-efficiency transformation method (Inoue et al., 1990, Gene 96:23-28). The protocol for library generation and display of library was used for lipase evolution.

Construction of Plasmids for Surface Display and Intracellular Expression of CMCase

For surface display of CMCase, the corresponding gene was subcloned into an Inp surface display vector, pSSTS 110, as described below. The 1.3-kb PCR products derived from pUBS 101 by using Pfu DNA polymerase (Stratagene, La Jolla, Calif.) with two primers (SEQ ID No. 1 and 2) were digested with XmaI and HindIII and then ligated with pSSTS 110 which had been digested with the same enzymes, generating pYSK3. This plasmid contains only the gene encoding the mature form of CMCase from amino acids 31 to 499 and lacks the signal sequence required for secretion. To achieve intracellular expression of the free form of CMCase, 1.3-kb DNA fragments of the CMCase gene were obtained by 30 cycles of PCR (94° C. for 30 s, 50° C. for 30 s, and 72° C. for 60 s) with two primers (SEQ ID No. 1 and 2). For correct translation of the CMCase gene in E. coli, the ATG start codon was added. PCR products were purified, digested with XmaI/HindIII, and ligated with pKK223-3 that had been digested with the same enzymes, resulting in a free-form expression vector, pYSK1.

Selection and Screening

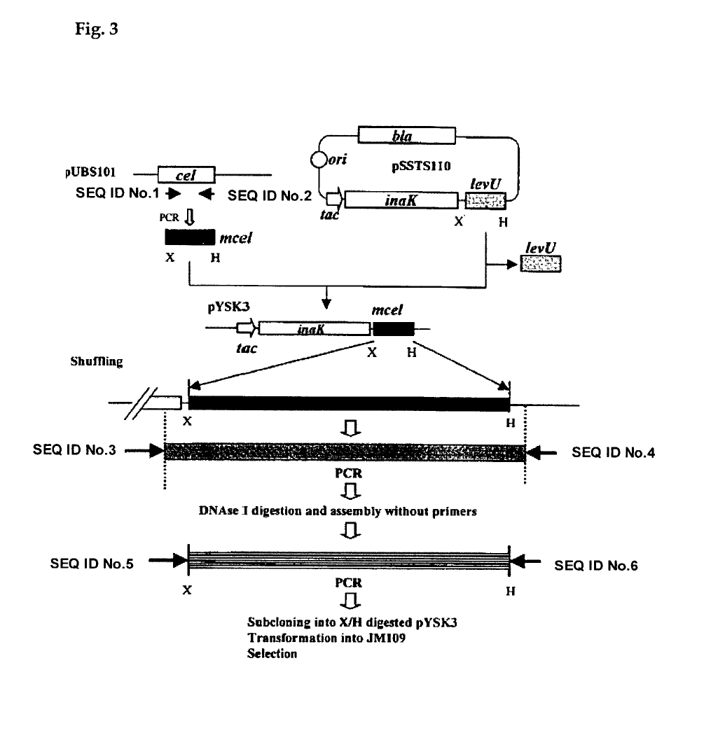

Transformants displaying a library of CMCase variants on the surface of E. coli cells were spread on M9 minimal medium plates containing 0.5% (wt/vol) CMC (Sigma), 1 mM of IPTG (Sigma), 100 g of thiamine per ml, and 50 mg of ampicillin per ml (M9-CMC plates). For the first positive selection, 150 larger colonies were picked up after a 72-h incubation at 37° C. and subsequently transferred onto an LB plate containing 100 μg of ampicillin per ml and 1 mM IPTG (LB-Amp-IPTG). Halo-forming activities of the cells were analyzed by the Congo red method (Park and Pack, 1986, Enzyme Microb. Technol. 8:725-728). After growth for 15 h at 37° C., bacterial colonies were overlaid with 10 ml of sterile top agar containing 0.5% CMC and then incubated at 37° C. for 6 h to allow hydrolysis of CMC. After this incubation, the plates were flooded with 0.2% (wt/vol) Congo red. After 30 min, the Congo red solution was poured off, and the plates were washed with 10 ml of 1 M NaCl for 10 min. Colonies that hydrolyze CMC were identified by yellow halos, where Congo red staining is absent (Park and Pack, 1986, Enzyme Microb. Technol. 8:725-728). During the first round of random mutagenesis and selection, 150 colonies were identified that showed higher growth rates and larger halos than control colonies containing the parent CMCase. FIG. 3. Colonies of E. coli JM109 displaying CMCase on their surfaces (mag-nification, 32.5). Parent CMCase (A) and the shuffled CMCase library (B) from the first round of mutagenesis are shown. Colonies were formed after 72 h of growth on M9 minimal agar plate at 37° C. containing 0.5% (wt/vol) CMC as the sole carbon source, 50 mg of ampicillin per ml, 100 mg of thiamine per ml, and 1 mM IPTG. (C) Transformants of the shuffled CMCase library were spread and grown for 24 h on M9 glucose agar plate at 37° C. These colonies were used for the next round of mutagenesis and selection; 1.3-kb fragments of evolved CMCases were amplified by colony PCR and then used as PCR templates for the next rounds. Colony PCR with YSK1 and YSK2 as primers was performed as described elsewhere (6) under PCR conditions of 30 cycles of 94° C. for 30 s, 65° C. for 30 s, and 72° C. for 60 s. Three rounds of random mutagenesis and screening were carried out, and 150 to 200 clones from each round were selected and characterized in detail.

CMCase Assay

Whole-cell and free-form CMCase activities were determined according to previous methods (Park and Pack, 1986, Enzyme Microb. Technol. 8:725-728). Enzymatic reactions were performed for 30 min at 37° C. with mixing. One unit of enzyme was defined as the quantity of enzyme capable of releasing 1 mmol of glucose equivalent per min.

Lipase Assay

Lipase activity was measured quantitatively by fluorescence spectrophotometry with coumarin oleate (Sigma, St. Louis, Mo.) as a substrate. Enzymatic reactions were kinetically assayed at room temperature with mixing.

SDS-PAGE and Western Blot Analysis

The expressed enzyme variants were analyzed by standard sodium dodecyl sulfate (SDS)-10% polyacrylamide gel electrophoresis (PAGE) and Western blot with rabbit anti-CMCase antibody. The soluble and insoluble fractions from total expressed CMCase were per-formed by centrifugation method. Briefly, 2.5 3 10 8 cells from 1 ml of culture (1.0 OD₆₀₀ nm) were pelleted by centrifugation at 12,000 rpm for 5 min. The cells were washed twice with 0.85% saline solution and resuspended in lysis buffer (50 mM Tris, 10 mM EDTA, 1 mMphenylmethylsulfonyl fluoride; pH 8.0). Then the cells were lyzed by ultrasonifier 450 (Branson, Danbury, Conn.). The soluble fraction was obtained from the supernatant after centrifugation at 12,000 rpm for 10 min, while the insoluble fraction was collected from the pellets.

DNA Sequencing

The 1.3-kb DNA fragment encoding the evolved CMCase and its flanking regions was sequenced in both forward and reverse directions by using a BigDye Terminator Ready-Reaction kit and an ABI Prism 377 DNA Sequencer (Perkin-Elmer/Applied Biosystems, Foster City, Calif.).

### Example 1

### Hydrolysis of CMC by CMCase

To examine whether the cells of E. coli, the host microbe used in this invention, are capable of utilizing hydrolyzed products of CMC by CMCase, a beta-glucosidase isolated from Bacillus subtilis, the hydrolyzed products were analyzed by High pH Anion Exchange Chromatography (HPAEC), one of Ion Chromatographies (Dionex Corp.), to which a Pulsed Amperometric Detection (PAD) system was attached. The column used was Carbopac PA1 Analysis (Dionex Corp.), deionized water was used as Eluent #1, 200 mM NaOH for Eluent #2, 200 mM NaOH, 1 M NaOAc for Eluent #3, and the flow rate was adjusted to 1 mL/min.

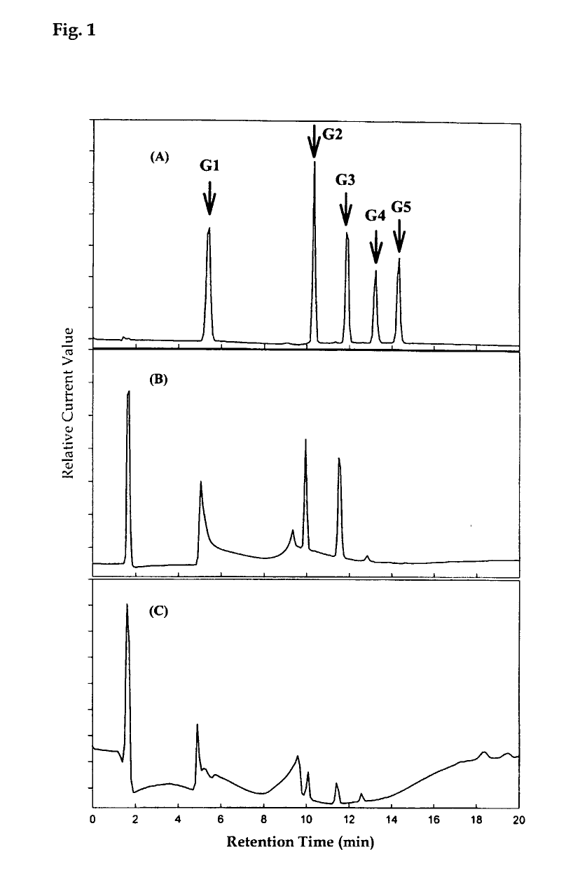

Further, glucose (G1) as a standard reagent, glucose dimer (G2), glucose trimer (G3), glucose tetramer (G4) and glucose pentamer (G5) were dissolved in distilled water with a concentration of 1 g/1L for each, respectively. The analysis was carried out according to the method described below and the results are shown in FIG. 1A.

Method is further described hereafter, first, partially purified CMCase was added to 0.5% CMC, dissolved in 50 mM buffered citrate solution, and was allowed to react at 55° C. for 15 hr. The product yielded was analyzed and the result showed that glucose, glucose dimer, glucose trimer and a small amount of glucose tetramer were produced (FIG. 1B), thus implying that the host microbe E. coli can proliferate by utilizing the glucose produced. Furthermore, the same result was obtained when new enzyme variant 2R59 was used, also produced under the same condition as described in this invention (FIG. 1C).

### Example 2

### Display of CMCase on E. coli Cell Surface

The INP surface display system developed by inventors of this invention was used to express CMCase on E. coli cell surface. First, oligonucleotides listed in SEQ ID No. 1 & 2 were used as primers to subclone CMCase gene into pSSTS 110, an INP displaying vector (KCTC 0327BP; Jung, et al., Nature Biotechnol., 1998, 16, 576-580), and performed a PCR using pUBS101 (Park et al., Agric. Biol. Chem., 1991, 55, 441-448) that carries CMCase gene as a main frame (DaKaRa ExTaq DNA polymerase, 30 cycles, 94° C., 1 min/50° C., 1 min/72° C., 1 min). Then, PCR-amplified 1.3 kb DNA fragments were digested with XmaI and HindIII and subcloned into pSSTS110, also treated with the same restriction enzymes, and the DNA fragments were then digested again with KpnI and HindIII and subcloned into pEIN229 (Jung et al., Nature Biotechnol., 1998, 16, 576-580) to construct a new plamid vector pYSK3 (FIG. 2). In FIG. 2, mCMCase indicates mature form of carboxymethylcellulase (CMCase) while INP represents the ice nucleation active protein. The pYSK3 was then transformed into E. coli JM 109 to generate a transformant JM 109/pYSK3. The transformant JM 109 was herewith named as Escherichia coli JM109/pYSK3, submitted to the gene bank in KRIBB (Korea Research Institute of Bioscience and Biotechnology) on Mar. 5, 1999 and the registration number KCTC 0584BP was assigned.

The nucleotide sequence of pYSK3 was determined by ABI Prism 377 DNA Sequencer (Perkin Elmer, USA) using Big Dye Terminator Kit (Perkin Elmer, USA). The nucleotide sequence of pYSK3 is shown in the SEQ ID No. 10 and its deduced amino acid sequence is shown in SEQ ID No. 10. When induced by an inducer after transforming into an E. coli host, the protein expressed was a fusion form of INP and mCMCase.

### Example 3

### Growth of E. coli Displaying CMCase on CMC as a Carbon Source

As shown in Example 1, the hydrolyzed products of CMC include a small amount of glucose that can be utilized by E. coli. To investigate whether host E. coli cells can grow on CMC as the only carbon source depending on the CMCase expressed on host E. coli cell surface, the plasmid pYSK3, constructed in Example 2, was transformed into E. coli JM109 and smeared on M9 minimal agar plates containing 0.5% (w/v) CMC, an inducer (1 mM IPTG; isopropyl-beta-D-glucopyranoside), 100 μg/mL Thiamine, 50 μg/mL ampicillin, and cultured at 37° C. for 72 hr and monitored the presence of any colony formation. As shown in FIG. 3, the transformant E. coli JM109/pYSK3 was shown to be able to grow and form colonies on the minimal agar plates described in the above.

### Example 4

### Library Construction of CMCase Gene and its Surface Display

The library of CMCase gene variants was constructed by DNA shuffling method by Stemmer (Stemmer, Nature, 1994, 370, 389-391). The process of DNA shuffling method is carried out in the following sequence: 1) the PCR amplification of a given gene and removal of primers, 2) the cleavage of amplified gene fragments by DnaseI and the separation of 50-100 bp nucleic acid fragments, 3) recombination of nucleic acid fragments by primer-less PCR and manufacture of the given gene by PCR using primers. The DNA shuffling method can be also used to construct a mutant library of a given gene using error-prone PCR, or artificial mutagenesis induced by recombination of nucleic acid fragments during the process of reassembly. The first shuffling step of CMCase gene in this invention is as follows. First, the starting nucleic acid fragments in shuffling were prepared by PCR amplification (30 cycles, 94° C., 1 min/50° C., 1 min/72° C., 1 min) of CMCase carried in pYSK3 produced in Example 2. by using two different primers; a 100 pmol primer of SEQ ID No. 3 and a 100 pmol primer of SEQ ID No. 4. The primers were then separated by electrophoresis on a 0.8% agarose gel and removed by using gene separation kit (QiaEx II DNA isolation & purification kit, QiaEx Corp., Germany), and the 1.3 kb DNA fragments were isolated and purified. Then 4μg of the 1.3 kb DNA fragments were digested with DNaseI, and 50-200 bp DNA fragments were isolated and purified by electrophoresis, and reassembly PCR was carried out (TaKaRa recombinant Taq DNA polymerase, 60 cycles; 94° C., 30 sec/50° C., 30 sec/72° C., 65 sec) without primers. The PCR product from the above reaction was then diluted 50 times and used for the final PCR (30 cycles; 94° C., 60 sec/55° C., 60 sec/72° C., 60 sec) template using a 30 pmol primer of SEQ ID No. 5 and a 30 pmol primer of SEQ ID No. 6, and as a result, obtained the shuffled 1.3 kb gene fragments of CMCase. The gene fragments were then subcloned into pYSK3 after digestion with XmaI and HindIII, and the recombinant plasmid was transformed into E. coli JM109 using high efficiency transformation method (Inoue et al., Gene, 1990, 96, 23-28), and finally the library of CMCase gene variants was constructed.

### Example 5

### Expression & Screening of Library of CMCase Gene Variants

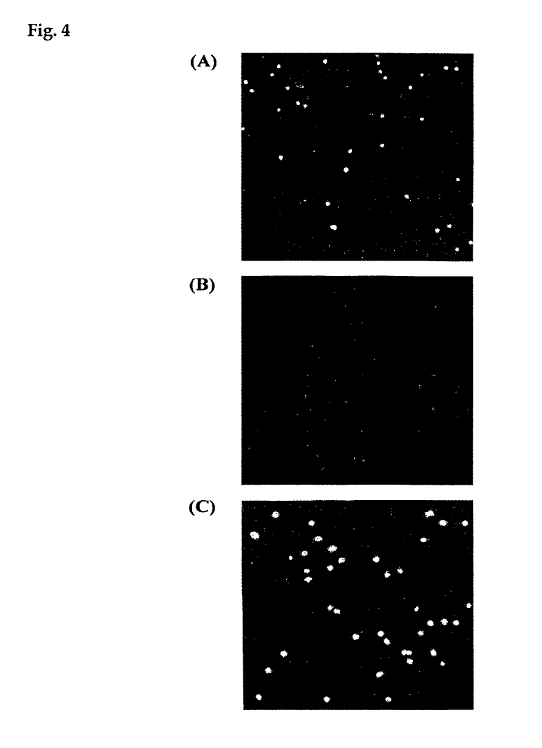

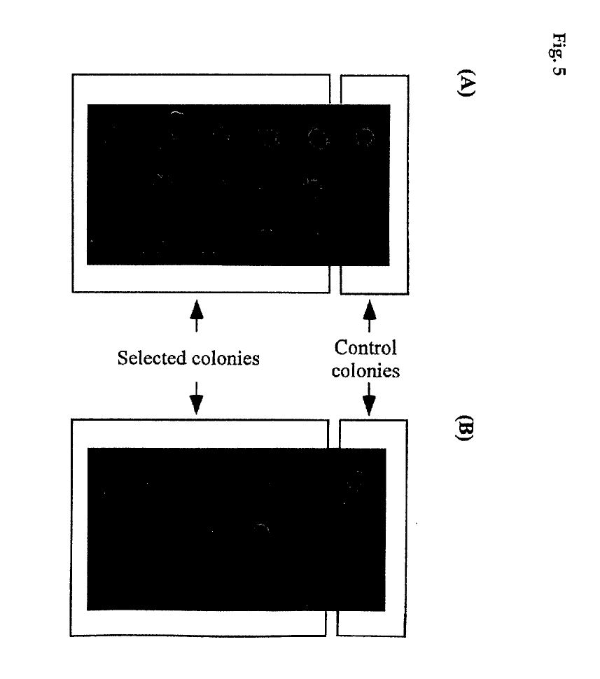

The CMCase gene variants prepared in Example 4 were smeared on CMC-containing M9 minimal agar plates and cultured at 37° C. for 72 hr in the same way as in Example 3, and approximately 200 large colonies with relatively fast growth rate were selected from approximately 10,000 variants by comparing the diameters of colonies (FIG. 4). The selected colonies were then compared again for reaffirmation by Congo Red staining method as described below. First, the selected 200 variants were subcultured over night on 100 μg/mL Luria-Bertani agar plates (LB medium, 5 g/L yeast extract, 10 g/L trypton and 5 g/L NaCl) with 1 mM IPTG, and then topped with liquid LB containing 0.75% top agar and 0.5% (w/v) CMC, and incubated at 37° C. for 6 hr for the hydrolysis of CMC. Then, the reducing sugars formed around variant colonies were stained with Congo Red dye for 30 min, washed in 1M NaCl for 10 min, and the formed halo sizes were measured again to ascertain the increased activity of CMCase expressed on E coli cell surface (FIG. 5).

### Example 6

### The Construction, Surface Expression, Screening & Selection of CMCase Variants by Utilization of the 2^{nd} and 3^{rd} DNA Shufflings

The 200 variants selected from the 1^{st} screening in Example 5 were used as substrates for the 2^{nd} DNA shuffling. Each colony of the 200 variant genes was transferred into a centrifuge tube, respectively, and after adding 10 μl of distilled water each tube was heated at 100° C. for the separation of DNA and centrifuged. The supernatant in each tube was recovered and used as DNA template, and only CMCase gene was amplified by PCR (30 cycles, 94° C., 1 min/50° C., 1 min/72° C., 1 min) using primers from SEQ ID No. 1 & 2 and PCR whole reaction kit (PCR premix, Bioneer Corp.). Then, another library of variants was constructed using the same method as in Example 4, and about 5 μg mixture of the above-mentioned 1.3 kb CMCase variant genes was used as a substrate for the 2^{nd} DNA shuffling. Subsequently, about 150 variants with increased CMCase activities were selected. By using the same method, variants by the 3^{rd} shuffling were produced and approximately 150 variants were finally selected.

### Example 7

### Comparison of Enzyme Activities of Stepwise Selected Variants Expressed on Bacterial Cell Surface

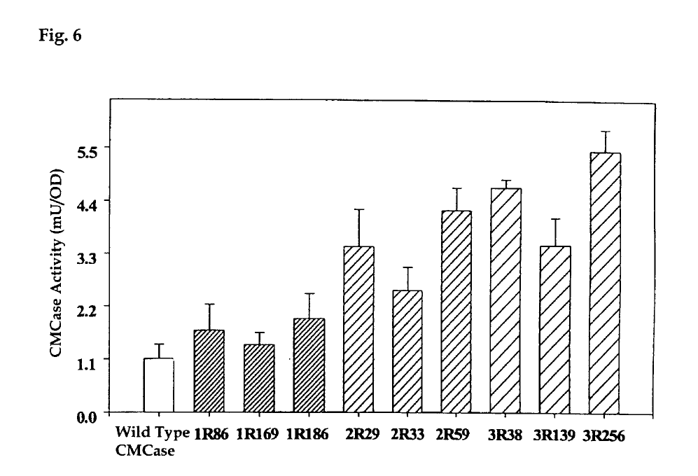

After comparing the enzyme activities of entire cells of 150-200 enzyme variants expressed on cell surfaces obtained from the 1 st, 2nd and 3rd screenings, respectively, 3 best clones were selected from each group. The enzyme activities of entire cells were measured as follows: first, E. coil variants were cultured at 37□ on LB liquid medium containing 100□/mL ampicillin and the turbidity was measured at 600 nm. When optical density was 0.4, protein expression was induced by 1 mM IPTG inducer for 1.5 hr. Then, 1 mL culture of E. coli, which was induced to express, was centrifuged and the cells were recovered. The cells were then washed in 0.05 M citrate buffer solution (pH 5.5), resuspended in 0.5 mL of the same buffer solution, mixed with 0.5 mL of 1% CMC solution, and incubated at 37□ for 30 min. Then the cells were removed by centrifugation, and the enzyme activities were examined by measuring the amount of reducing sugar separated in the supernatant by DNS (Park et al., Agric. Biol. Chem., 1991, 55, 441-448). Finally, 3 best clones were selected from each step, respectively. The 3 best variants selected from each group were named as follows: 1 R86, 1R69, 1R186 from the 1st screening; 2R29, 2R33, 2R59 from the 2nd screening; and 3R38, 3R139, 3R256 from the 3rd screening, and their entire cell activities are shown in FIG. 6. Optical density was measured at 600 nm, and 3R256, a variant selected from the 3rd screening was shown to have a 4-fold increase in entire cell activity.

### Example 8

### Comparison of Protein Expression and Enzyme Activities of Purified Enzyme Variants Selected from Each Step

To examine whether enzyme variants selected in Example 7 can still show good activities when expressed in a form separated from INP, each enzyme variant gene was subcloned into an expression vector pKK223-3 (Pharmacia, Sweden) for the independent expression. First, each enzyme variant gene was amplified as in Example 2 using primers of SEQ ID No. 6 & No. 7, digested with XmaI and HindIII, and subcloned into a vector pKK233-3, which was also treated with the same restriction enzymes. Then, each recombinant plasmid generated was transformed into E. coli JM 109 and induced to express by 1 mM IPTG on LB plate. To measure the enzyme activities expressed within cells, cells were lysed and enzyme activities were measured in the same way as in the measurement of entire cell activities in Example 7, and the total amount of protein produced was determined by Bradford kit (Biorad, USA).

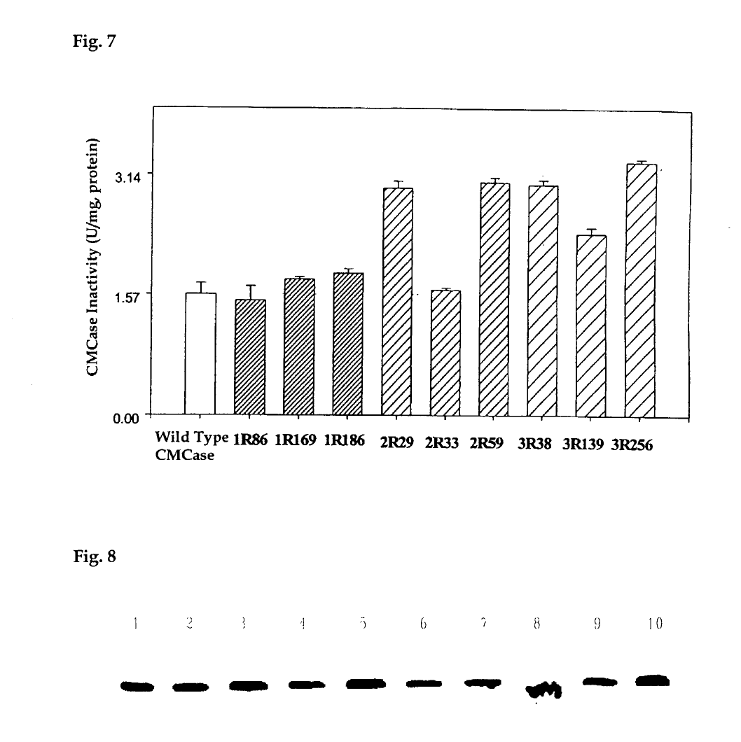

The non-enzyme activities of CMCase were measured and shown in FIG. 7. As shown in Example 7, variants 2R29, 2R59, 3R38, and 3R256 showed a two-fold increase in activities compared to those of wild type enzymes, unlike the entire cell activities shown in Example 7.

### Example 9

### Sequence Comparison of Nucleotides & Amino Acids of Enzyme Variants

DNA sequence change resulting from mutagenesis is summarized as follows. The nucleotide sequences of 9 enzyme variant genes selected from Example 7 & 8, were determined by ABI prism 377 DNA sequencer (Perkin Elmer, USA) using Big Dye Terminator kit (Perkin Elmer, USA), and the results of varied amino acids are shown in Table 1.

TABLE 1

| Transformants | Substituted amino acids |
|---|---|
| 1R86 | K*45E**, A55R, R83W, 1191T, N223Y, T256A, |
| | Q350R, N381D |
| 1R169 | F91L, I188V, S335L, T342A, T481S |
| 1R186 | G123E, I1339V, N415D |
| 2R29 | A222V, S308P, L315M, S335L, A360L, A360S, |
| | N368Y, A397T |
| 2R33 | N391, K60R, N244S, G267D, Q357R, 1370V, T3821 |
| 2R59 | D317N, K347N |
| 3R38 | K109N, in a form disconnected from the 369th amino |
| | acid |
| 3R139 | K109N, T260A, T2851, K347R, Y368H, H424Y, |
| | A430P |
| 3R256 | V901, T260A, T2851, Y358H, H424Y, A430P |

*represents amino acid of wild type CMCase
** represents substituted amino acid of variant enzyme

As shown in Table 1, each variant underwent random mutagenesis, and there was not a common site for the substitution of amino acids, but it differed from variant to variant. In 3R38, though having only the active part of the enzyme and with the CMC binding part disconnected, the variant showed relatively high enzyme activity.

### Example 10

### Surface Display of Lipase

Surface display on E. coli of a thermostable lipase from Pseudomonas fluorescens SIK W1 is summarized as follows. The general procedure of surface display for lipase followed the same protocol for CMCase display described in detail in Example 2. The INP surface display system developed by inventors of this invention was used to express lipase on E. coli cell surface.

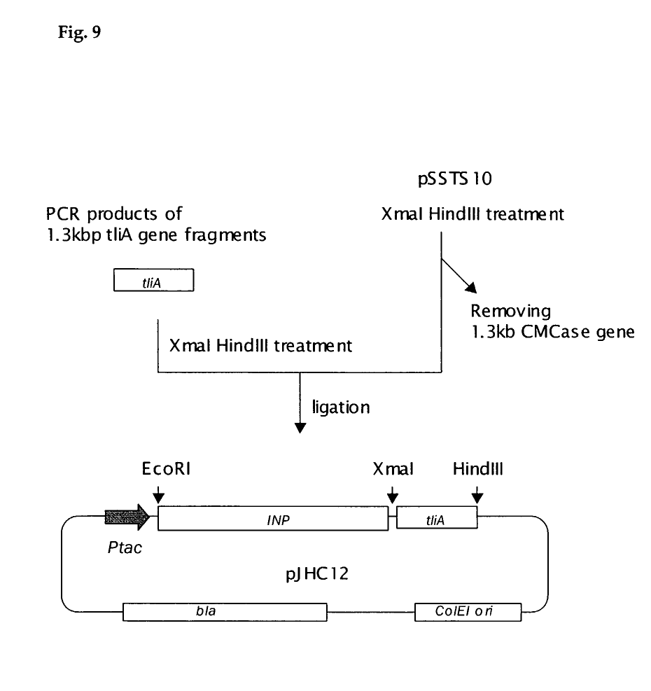

The gene sequence tliA from gene cluster of thermostable lipase and its ABC(ATP Binding Cassette) transporter (GenBank accession number, AF083061), encoding lipase, was PCR cloned. First, oligonucleotides listed in SEQ ID No. 8 & 9 were used as primers to subclone lipase gene into pSSTS 110, an INP displaying vector (KCTC 0327BP; Jung, et al., Nature Biotechnol., 1998, 16, 576-580), and performed a PCR using pHOPE (Ahn et al., J. Bacteriol., 181:1847-1852(1999)) as a PCR template that carries thermostable lipase gene as a main frame (DaKaRa ExTaq DNA polymerase, 30 cycles, 94° C., 1 min/50° C., 1 min/72° C., 1 min). Then, PCR-amplified 1.3 kb DNA fragments were digested with XmaI and HindIII and subcloned into pSSTS110, also treated with the same restriction enzymes, and the DNA fragments were then digested again with KpnI and HindIII and subcloned into pEIN229 (Jung et al., Nature Biotechnol., 1998, 16, 576-580) to construct a new plamid vector pJHC12. In FIG. 9, TliA indicates thermostable lipase while INP represents ice nucleation active protein. The pJHC 12 was then transformed into E. coli TG 1, a commonly used in the laboratory, to generate a transformant TG1. The transformant TG1 was herewith named as Escherichia coli TG1/pJHC12.

The nucleotide sequence of pJHC12 was determined by ABI Prism 377 DNA Sequencer (Perkin Elmer, USA) using Big Dye Terminator Kit (Perkin Elmer, USA). The nucleotide sequence encoding the INP-TliA fusion protein from pJHC12 and its deduced amino acid sequence is shown in the SEQ ID No. 11. When induced by an inducer after transforming into an E. coli host, the protein expressed was a fusion form of both INP and lipase by analyzed by SDS-PAGE.

### Example 11

### Surface Display of Lipase Library

The library of lipase gene was generated by DNA shuffling method (Stemmer, Nature, 1994, 370, 389-391) as described in Example 4 of CMCase library generation. The first shuffling step of lipase gene in this invention is as follows. First, the starting nucleic acid fragments in shuffling were prepared by PCR amplification (30 cycles, 94° C., 1 min/50° C., 1 min/72° C., 1 min) of lipase carried in pJHC 12 produced in Example 10 by using two different primers; a 100 pmol primer of SEQ ID No. 3 and a 100 pmol primer of SEQ ID No. 4. The amplified DNA fragments were then separated by electrophoresis on a 0.8% agarose gel and removed by using gene separation kit (QiaEx II DNA isolation & purification kit, QiaEx Corp., Germany), and the 1.3 kb DNA fragments were isolated and purified. Then 4 μg of the 1.3 kb DNA fragments were digested with Dnase I, and 50-200 bp DNA fragments were isolated and purified by electrophoresis, and reassembly PCR was carried out (TaKaRa recombinant Taq DNA polymerase, 60 cycles; 94° C., 30 sec/50° C., 30 sec/72° C., 65 sec) without primers. The PCR product from the above reaction was then diluted 50 times and used for the final PCR (30 cycles; 94° C., 60 sec/55° C., 60 sec/72° C., 60 sec) template using a 30 pmol primer of SEQ ID No. 5 and a 30 pmol primer of SEQ ID No. 6, and as a result, obtained the shuffled 1.3 kb gene fragments of thermostable lipase. The gene fragments were then subcloned into pJHC12 after digestion with XmaI and HindIII, and the recombinant plasmid was transformed into E. coli TG1 using high efficiency transformation method (Inoue et al., Gene, 1990, 96, 23-28), and finally the library of lipase gene was constructed.

### Example 12

### Library Screening of Displayed Lipase Library

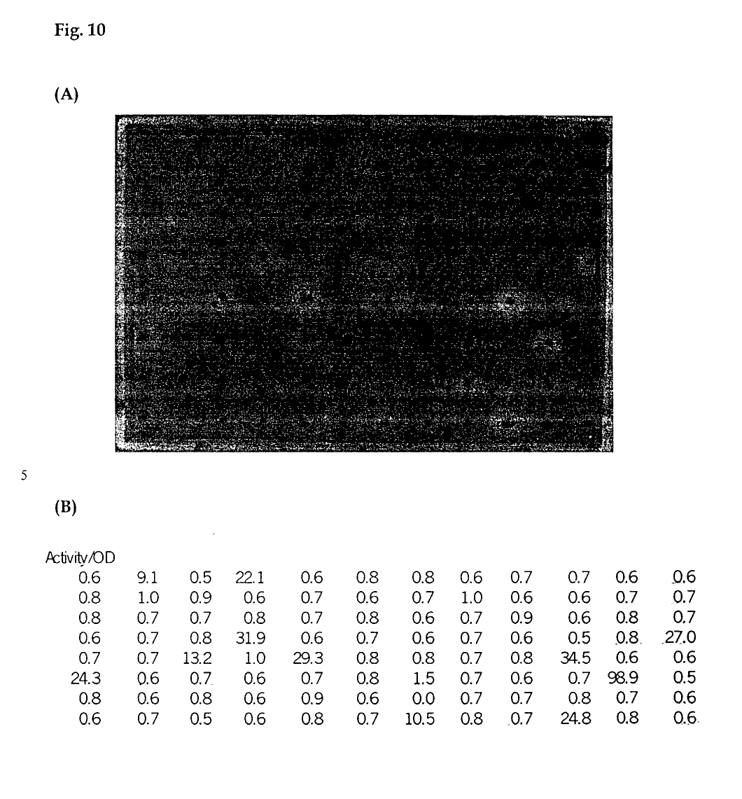

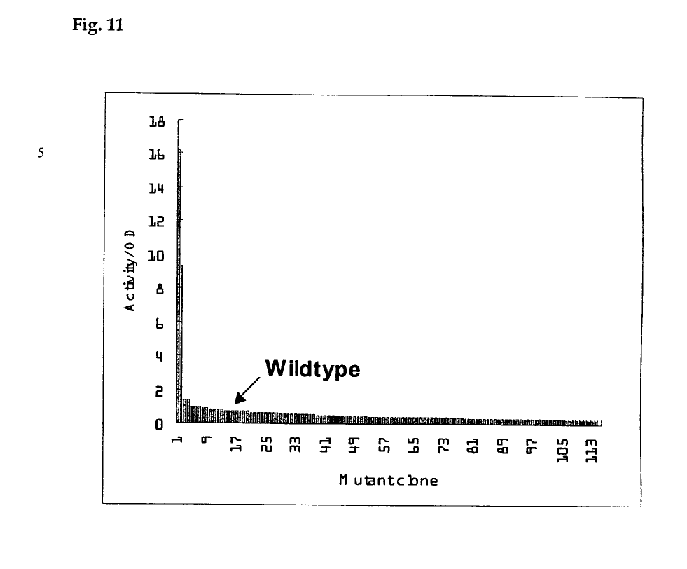

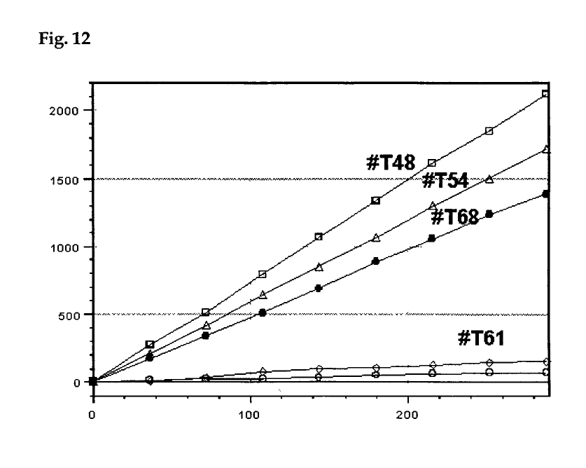

The population of bacteria displaying lipase gene variants prepared in Example 11 was spread on LB agar plate containing 100 mg/ml of ampicillin and was incubated overnight at 37° C. And then for first screening, each colonies were transferred to 0.5% (weight/vol.) tributyrin-LB agar plate with automatic colony picker (Q-Pix, Genetix, UK) and said plates were incubated overnight at 25° C. for induction of INP-TliA fusion protein sysnthesis. Halo size around the colonies on tributyrin LB agar plate was proportionally related to the activity of lipase displayed on the surface of E. coli, which was confirmed by measuring whole cell activity of lipase as shown FIG. 10. From more than 25,000 colonies, 120 colonies were selected from tributyrin LB agar plate according to the halo size and then further confirmed by directly measuring whole cell activity of lipase in 96 well plate with a fluorescent substrate, coumarin fluorescein as shown in FIG. 11. Finally, 4 clones (TG48, TG54, TG61, and TG68) were further confirmed in 96 well plate with a fluorescent substrate, coumarin fluorescein as shown in FIG. 12, were selected and their plasmid DNA were purified. Table 2 shows their nucleotide and amino acid substitution.

TABLE 2
Nucleotide and amino acid changes of selected TliA mutants

| | DNA Changes | Amino acids changes |
|---|---|---|
| TG48 | A23G, A40G | N8S,S14G |
| | TI69C,T214A | W72R |
| | A1098G, T1248C | |
| TG54 | A1081 T, A1098G | N361Y |
| TG68 | C732T, T8058C, C67G, | S269P |
| | A1098G | T323M, Q356R |
| TG61 | T71C,A1098G | 124T |

What is claimed is:

1. A method of selecting a bacterium comprising a nucleic acid sequence encoding a polypeptide capable of catalyzing a chemical reaction from a plurality of candidate bacteria comprising the following steps of:
(a) generating a pool of nucleic acids by introducing at least one nucleotide change into the target nucleic acids encoding the polypeptide capable of catalyzing the desired chemical reaction,
(b) constructing library vectors to be transformed into a host cell after subcloning said pool of candidate nucleic acids into a surface display vector wherein said resulting vectors direct expression of fusion polypeptides of display motifs and candidate polypeptides and said fusion polypeptides are to be anchored to the surface of said bacteria,
(c) transforming said library vectors into bacteria,
(d) expressing each of said fusion polypeptides on the surface of host bacteria, and
(e) selecting a bacterium expressing a desired polypeptide on the basis of said host bacterial phenotypic changes

2. A method of selecting a bacterium comprising a nucleic acid sequence encoding a polypeptide capable of catalyzing a chemical reaction from a plurality of candidate bacteria comprising the following steps of:
(a) generating a pool of nucleic acids by introducing at least one nucleotide change into the target nucleic acids encoding the polypeptide capable of catalyzing the desired chemical reaction,
(b) constructing library vectors to be transformed into a host cell after subcloning said pool of candidate nucleic acids into a surface display vector wherein said resulting vectors direct expression of fusion polypeptides of display motifs and candidate polypeptides and said fusion polypeptides are to be anchored to the surface of said bacteria,
(c) transforming said library vectors into bacteria,
(d) expressing each of said fusion polypeptides on the surface of host bacteria, and
(e) selecting a bacterium expressing a desired polypeptide on the basis of visual changes of substrates for said chemical reaction.

3. The method of claims 1 and 2, wherein said host organism is selected from the group comprising Gram negative bacteria, Gram positive bacteria, yeast, fungi, mammalian cells, or spores.

4. The method of claim 3, wherein said host cell is Escherichia coli.

5. The method of claims 1 and 2, wherein said display motif is selected from the group of surface proteins of said host organism.

6. The method of claim 5, wherein said display motif is an ice-nucleation protein from Pseuomdonas syringae.

7. The method of claims 1 and 2, wherein said catalytic polypeptide is selected from the group of enzymes.

8. The method of claim 7, wherein said enzyme is selected from the group consisting of oxidoreductase, transferase, hydrolase, lyase, isomerase, and ligase.

9. The method of claim 8, wherein said enzyme is a polymer hydrolase.

10. The method of claim 9, wherein said hydrolase is a cellulase.

11. The method of claim 9, wherein said hydrolase is a lipase.

12. The method of claim 1 and 2, wherein said catalytic polypeptide is selected from the group of catalytic antibodies.

13. The method of claim 1, further defined as comprising selecting a bacterium whose phenotypic change is based on the different growth rate.

14. The method of claim 13, wherein said different growth rate is based on the different colony size on the semisolid surface.

15. The method of claim 14, wherein said semisolid surface is an agar plate.

16. The method of claim 13, wherein said different growth rate is based on the change of optical density in liquid culture.

17. The method of claim 13, wherein said host bacterial growth is supported by products released from catalysis of substrate.

18. The method of claim 2, wherein said visual change of substrates is clearance around the bacterial colonies.

19. The method of claim 18, wherein said clearance is from the hydrolysis of polymer substrate.

20. The method of claim 2, wherein said visual change of products is turbidity of substrate.

21. The method of claim 2, wherein said visual change of substrates is fluorescence.

22. The method of claim 2, wherein said visual change of substrates is color change of chromogenic substrate.

23. The method of claim 2, wherein said substrate is selected from the group of polymers comprising carbohydrate polymers, lipid, polypeptides, and synthetic organic polymers.

24. The method of claim 23, wherein said polymer is selected from the group comprising cellulose, carboxymethylcellulose, starch, xyllan, pullulan, chitin, chitosan, dextran, levan, curdlan, extracted oil from plants, casein, and/or soy protein.

25. A population of organisms comprising a nucleic acid sequence encoding a polypeptide capable of catalyzing a chemical reaction comprising the following steps of:
(a) generating a pool of nucleic acids by introducing at least one nucleotide change into the target nucleic acids encoding the polypeptide capable of catalyzing the desired chemical reaction,
(b) constructing library vectors to be transformed into a host organism after subcloning said pool of candidate nucleic acids into a surface display vector wherein said resulting vectors direct expression of fusion polypeptides of display motifs and candidate polypeptides and said fusion polypeptides are to be anchored to the surface of said organism,
(c) transforming said library vectors into host organisms, and
(d) expressing each of said fusion polypeptides on the surface of host organism.

26. The method of claim 25, wherein said host organism is selected from the group comprising Gram negative bacteria, Gram positive bacteria, yeast, fungi, mammalian cells, or spores.

27. The method of claim 26, wherein said host cell is Escherichia coli.


SEQUENCE LISTING (11 sequences)

SEQ ID NO: 1 - DNA, 24 bp, Bacillus subtilis
gcatcagcacccgggacaaaaacg

SEQ ID NO: 2 - DNA, 24 bp, Bacillus subtilis
gctcggtacaagcttatcatataa

SEQ ID NO: 3 - DNA, 24 bp, Bacillus subtilis
gacgaggacgatgactggatagag

SEQ ID NO: 4 - DNA, 24 bp, Bacillus subtilis
ctgaaaatcttctctcatccgcca

SEQ ID NO: 5 - DNA, 24 bp, bacillus subtilis
gacgatgactggatagaggtaaag

SEQ ID NO: 6 - DNA, 24 bp, bacillus subtilis
atcttctctcatccgccaaaacag

SEQ ID NO: 7 - DNA, 31 bp, Bacillus subtilis
ggtaaagcccgggatgacaaaaacgccagta

SEQ ID NO: 8 - DNA, 29 bp, Bacillus sbutilis
aagagagaccccgggatgggtgtatttga

SEQ ID NO: 9 - DNA, 29 bp, Bacillus subtilis
tacacctcgcaggtaaagcttaaacgcat

SEQ ID NO: 10 - protein, 1621 aa, BACILLUS SUBTILIS
MTLDKALVLRTCANNMADHCGLIWPASGTVESRYWQSTRRHENGLVGLLWGAGTSAFLSVHADARWIVCEVAVADIISLEEPGMVKFPRAEVVHVGDRISASHFISARQADPASTSTSTSTSTLTPMPTAIPTPMPAVASVTLPVAEQARHEVFDVASVSAAAAPVNTLPVTTPQNLQTATYGSTLSGDNHSRLIAGYGSNETAGNHSDLIAGYGSTGTAGYGSTQTSGEDSSLTAGYGSTQTAQEGSNLTAGYGSTGTAGSDSSLIAGYGSTQTSGGDSSLTAGYGSTQTAQEGSNLTAGYGSTGTAGVDSSLIAGYGSTQTSGSDSALTAGYGSTQTAQEGSNLTAGYGSTGTAGSDSSLIAGYGSTQTSGSDSSLTAGYGSTQTAQEGSNLTAGYGSTGTAGVDSSLIAGYGSTQTSGSDSALTAGYGSTQTAQEGSNLTAGYGSTGTAGADSSLIAGYGSTQTSGSESSLTAGYGSTQTAREGSTLTAGYGSTGTAGADSSLIAGYGSTQTSGSESSLTAGYGSTQTAQQGSVLTSGYGSTQTAGAASNLTTGYGSTGTAGHESFIIAGYGSTQTAGHKSILTAGYGSTQTARDGSYLIAGYGSTGTAGSGSSLIAGYGSTQTASYRSMLTAGYGSTQTAREHSDLVTGYGSTSTAGSNSSLIAGYGSTQTAGFKSILTAGYGSTQTAQERSDLVAGYGSTSTAGYSSSLIAGYGSTQTAGYESTLTAGYGSTQTAQENSSLTTGYGSTSTAGYSSSLIAGYGSTQTAGYESTLTAGYGSTQTAQERSDLVTGYGSTSTAGYASSLIAGYGSTQTAGYESTLTAGYGSTQTAQENSSLTTGYGSTSTAGFASSLIAGYGSTQTAGYKSTLTAGYGSTQTAEYGSSLTAGYGSTATAGQDSSLIAGYGSSLTSGIRSFLTAGYGSTLIAGLRSVLIAGYGSSLTSGIRSTLTAGYGSNQIASYGSSLIAGHESIQVAGNKSMLIAGKGSSQTAGFRSTLIAGAGSVQLAGDRSRLIAGADSNQTAGDRSKLLAGNNSYLTAGDRSKLTGGHDCTLMAGDQSRLTAGKNSVLTAGARSKLIGSEGSTLSAGEDSTLIFRLWDGKRYRQLVARTGENGVEADIPYYVNEDDDIVDKPDEDDDWIEVKPGTKTPVAKNGQLSIKGTQLVNRDGKAVQLKGISSHGLQWYGEYVNKDSLKWLRDDWGITVFRAAMYTADGGYIDNPSVKNKMKEAVEAAKELGIYVIIDWHILNDGNPNQNKEKAKEFFKEMSSLYGNTPNVIYEIANEPNGDVNWKRDIKPYAEEVISVIRKNDPDNIIIVGTGTWSQDVNDAADDQLKDANVMDALHFYAGTHGQFLRDKANYALSKGALIFVTEWGTSDASGNGGVFLDQSREWLKYLDSKTISWVNWNLSDKQESSSALKPGASKTGGWRLSDLSASGTFVRENILGTKDSTKDIPETPAKDKPTQENGISVPYRAGDGSMNSNQIRPQLQIKNNGNTTVDLKDVTARYWYNAKNKGQNVDCDYAHVGCGNVTYKFVTLHKPKQGADTYLELGFKNGTLAPGASTGNIQLRLHNDDWSNYAQSGDYSFFKSNTFKTTKKITLYDKLGCFWRMREDFSA

SEQ ID NO: 11 - protein, 1626 aa, Bacillus subtilis
MTLDKALVLRTCANNMADHCGLIWPASGTVESRYWQSTRRHENGLVGLLWGAGTSAFLSVHADARWIVCEVAVADIISLEEPGMVKFPRAEVVHVGDRISASHFISARQADPASTSTSTSTSTLTPMPTAIPTPMPAVASVTLPVAEQARHEVFDVASVSAAAAPVNTLPVTTPQNLQTATYGSTLSGDNHSRLIAGYGSNETAGNHSDLIAGYGSTGTAGYGSTQTSGEDSSLTAGYGSTQTAQEGSNLTAGYGSTGTAGSDSSLIAGYGSTQTSGGDSSLTAGYGSTQTAQEGSNLTAGYGSTGTAGVDSSLIAGYGSTQTSGSDSALTAGYGSTQTAQEGSNLTAGYGSTGTAGSDSSLIAGYGSTQTSGSDSSLTAGYGSTQTAQEGSNLTAGYGSTGTAGVDSSLIAGYGSTQTSGSDSALTAGYGSTQTAQEGSNLTAGYGSTGTAGADSSLIAGYGSTQTSGSESSLTAGYGSTQTAREGSTLTAGYGSTGTAGADSSLIAGYGSTQTSGSESSLTAGYGSTQTAQQGSVLTSGYGSTQTAGAASNLTTGYGSTGTAGHESFIIAGYGSTQTAGHKSILTAGYGSTQTARDGSYLIAGYGSTGTAGSGSSLIAGYGSTQTASYRSMLTAGYGSTQTAREHSDLVTGYGSTSTAGSNSSLIAGYGSTQTAGFKSILTAGYGSTQTAQERSDLVAGYGSTSTAGYSSSLIAGYGSTQTAGYESTLTAGYGSTQTAQENSSLTTGYGSTSTAGYSSSLIAGYGSTQTAGYESTLTAGYGSTQTAQERSDLVTGYGSTSTAGYASSLIAGYGSTQTAGYESTLTAGYGSTQTAQENSSLTTGYGSTSTAGFASSLIAGYGSTQTAGYKSTLTAGYGSTQTAEYGSSLTAGYGSTATAGQDSSLIAGYGSSLTSGIRSFLTAGYGSTLIAGLRSVLIAGYGSSLTSGIRSTLTAGYGSNQIASYGSSLIAGHESIQVAGNKSMLIAGKGSSQTAGFRSTLIAGAGSVQLAGDRSRLIAGADSNQTAGDRSKLLAGNNSYLTAGDRSKLTGGHDCTLMAGDQSRLTAGKNSVLTAGARSKLIGSEGSTLSAGEDSTLIFRLWDGKRYRQLVARTGENGVEADIPYYVNEDDDIVDKPDEDDDWIEVKPGMGVFDYKNLGTEASKTLFADATAITLYTYHNLDNGFAVGYQQHGLGLGLPATLVGALLGSTDSQGVIPGIPWNPDSEKAALDAVHAAGWTPISASALGYGGKVDARGTFFGEKAGYTTAQAEVLGKYDDAGKLLEIGIGFRGTSGPRESLITDSIGDLVSDLLAALGPKDYAKNYAGEAFGGLLKTVADYAGAHGLSGKDVLVSGHSLGGLAVNSMADLSTSKWAGFYKDANYLAYASPTQSAGDKVLNIGYENDPVFRALDGSTFNLSSLGVHDKAHESTTDNIVSFNDHYASTLWNVLPFSIANLSTWVSHLPSAYGDGMTRVLESGFYEQMTRDSTIIVANLSDPARANTWVQDLNRNAEPHTGNTFIIGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS